(19) United States
(12) Patent Application Publication
(10) Pub. No.: US 2006/0058262 A1
(43) Pub. Date: Mar. 16, 2006

(54) New injectable formulations containing progesterone

(75) Inventors: Giorgio Zoppetti, Milano (IT); Marco Pizzutti, Malnate (IT); Nadia Puppini, Como (IT)

Correspondence Address: ABELMAN, FRAYNE & SCHWAB, 666 THIRD AVENUE, 10TH FLOOR, NEW YORK, NY, 10017, US

(73) Assignee: ALTERGON S.A.

(21) Appl. No.: 11/229,212

(22) Filed: Sep. 16, 2005

(30) Foreign Application Priority Data
Sep. 16, 2004 (IT) MI2004A001763

(51) Int. Cl. A61K 31/724; A61K 31/57

(52) U.S. Cl. 514/58; 514/171

(57) ABSTRACT

The present invention relates to injectable progesterone formulations and processes for their preparation.

### FIELD OF THE INVENTION

The present invention relates to new progesterone formulations for injectable use.

### STATE OF THE ART

Progesterone (Pregn-4-ene-3,20-dione) presents numerous pharmacological applications. This hormone is used, for example, in assisted reproduction protocols, in the treatment of threatened miscarriage and for preventing habitual miscarriage.

In threatened miscarriage therapy, high plasma levels of progesterone, at least 200 nmol/ml, must be attained and maintained in order to achieve a therapeutic effect.

However, because of the slow gastrointestinal absorption and high hepatic metabolism of this hormone, such plasma levels of progesterone cannot be achieved by oral administration. Therefore, in the treatment of the aforesaid pathologies, progesterone is generally administered intramuscularly.

Because of the lipophilicity of progesterone, commercially available injectable formulations of this hormone are in the form of dispersions micronized in ethyl oleate. However, these types of formulations present a number of drawbacks: they are not well tolerated as they give rise to irritations at the injection site, they cannot be administered subcutaneously, this being the more easy and more danger-free route, and furthermore they make it necessary to administer high doses of progesterone, in the order of 100 mg per day, to obtain adequate plasma levels. On the other hand, solving said problems by administering aqueous solutions of synthetic or semisynthetic progestinic drugs has proved to be inadvisable, as these products often present adverse effects.

There is therefore a strongly felt need in the art for injectable aqueous solutions of progesterone.

It has been known for some time, for example from U.S. Pat. No. 4,727,064 and WO85/02767, that the water solubility of poorly water-soluble drugs such as progesterone can be increased by the formation of a complex with β-cyclodextrin ethers, specifically hydroxypropyl-β-cyclodextrin. In the aforesaid patents the suitability of these formulations for preparing injectable solutions is stated.

Although the aforesaid patents have now been granted for some years, there is currently no commercially available formulation for parenteral administration in the form of an aqueous solution comprising complexes of hydroxypropyl-β-cyclodextrin with progesterone. This suggests a considerable difficulty in the practical implementation of these types of formulations conceived on paper, made further apparent by the considerable need felt in the art.

The present inventors have prepared aqueous solutions of the hydroxypropyl-β-cyclodextrin-progesterone complex by following the teachings of the known art and have in fact found that these solutions are not stable over time and are therefore unusable by injection.

Specifically, if the solutions obtained after dissolving the complex are left to stand at ambient temperature, they form a precipitate after a period of about 24 hours. Even if the precipitate is removed by filtration, a precipitate re-forms anyway in the clear solution thus obtained after about 48 hours.

This phenomenon is not described in any of the aforementioned patents.

The precipitate does not dissolve in water, 96% EtOH, aqueous solutions containing propylene glycol or polysorbates such as Tween 20. The problem observed therefore makes it totally impossible to use the formulations described in the known art by means of injection.

### SUMMARY OF THE INVENTION

The present inventors have now surprisingly found that the formation of a precipitate from solutions of progesterone-hydroxypropyl-β-cyclodextrin complexes is related to the presence of unsubstituted β-cyclodextrin impurities in commercially available hydroxypropyl-β-cyclodextrin preparations for pharmaceutical use. The observed precipitation is surprising and unexpected particularly in the light of the water solubility of unsubstituted β-cyclodextrin which the known art states to be 1.8 g/100 ml, and of the small quantities thereof present in preparations of hydroxypropyl-β-cyclodextrin (usually not greater than 1%).

The present inventors have also found that precipitate formation can be avoided if solutions of the hydroxypropyl-β-cyclodextrin-progesterone complex containing a quantity of unsubstituted β-cyclodextrin not less than 0.3%, and preferably less than 0.1%, are prepared. These solutions are actually stable for at least 40 days at 25° C. and are suitable for use by injection.

### DETAILED DESCRIPTION OF THE INVENTION

Therefore, a first aspect of the present invention is the provision of injectable progesterone formulations comprising the hydroxypropyl-β-cyclodextrin-progesterone complex characterised by containing a quantity of unsubstituted β-cyclodextrin less than 0.3% and preferably less than 0.1% w/w on the quantity of hydroxypropyl-β-cyclodextrin.

The aforesaid formulations can be in the form of a ready-to-use aqueous solution or lyophilizate which is reconstituted in water when required to provide an extemporaneous injectable aqueous solution.

As will be demonstrated in examples 1,2,3 and 5 to follow, the aqueous solutions of the present invention are stable over time, without any precipitate forming for at least 40 days at 25° C. and at least 48 hours at 5° C., being therefore suitable for use by injection.

Moreover, the solutions of the present invention can also contain very high concentrations of progesterone.

Therefore, the formulations of the present invention are particularly suitable for intramuscular and especially subcutaneous use, where small amounts of solution have to be used.

Hydroxypropyl-β-cyclodextrin, obtained by the propylation of β-cyclodextrin hydroxyls, is commercially available with various degrees of substitution which indicate the average number of hydroxypropyl groups per cyclodextrin. Commercial hydroxypropyl-β-cyclodextrin preparations contain impurities consisting of unsubstituted β-cyclodextrin whose quantity usually increases with decreasing degree of substitution of hydroxypropyl-β-cyclodextrin.

In accordance with a first embodiment, the formulations of the present invention can be obtained by preparing, following the methods already known to experts of the art, progesterone-hydroxypropyl-β-cyclodextrin complexes from preparations of hydroxypropyl-β-cyclodextrin containing a quantity of unmodified β-cyclodextrin less then 0.3%, preferably less than 0.1%, on the quantity of hydroxypropylated-β-cyclodextrin as described in example 3.

However, all the hydroxypropyl-β-cyclodextrin preparations that are listed in the Pharmacopoeia and hence suitable for pharmaceutical use contain unsubstituted β-cyclodextrin impurities in quantities greater than the aforesaid values.

Therefore, in accordance with an alternative and particularly preferred embodiment, the formulations of the present invention can also be obtained starting from hydroxypropyl-β-cyclodextrin preparations that contain quantities of unsubstituted β-cyclodextrin greater than the aforesaid values, by means of a particular process established by the present inventors which enables the β-cyclodextrin quantity to be decreased to less than 0.3% w/w and preferably to less than 0.1% w/w on the quantity of hydroxypropylated β-cyclodextrin.

This process comprises the following steps:

a) preparing an aqueous solution of hydroxypropyl-β-cyclodextrin having a concentration between 12% and 55% w/w and preferably 48% w/w;

b) adding micronized progesterone under stirring to the solution of step a), preferably in a quantity equal to 10% by weight on the quantity of hydroxypropyl-β-cyclodextrin, corresponding to the saturation concentration of the solution, and optionally filtering, preferably with a 0.45 μm filter, to obtain a transparent colourless solution;

c) maintaining the solution at a temperature between 2 and 8° C., preferably 5° C., for a period of at least 2 days, preferably between 2 and 10 days, preferably between 3 and 8 days and even more preferably 3 days, to hence obtain a precipitate;

d) carrying out a clarification filtration using a filter with a porosity between 0.45 and 0.8 micron.

Usually, in order to achieve complete dissolution of progesterone, micronized progesterone needs to be used in step b), and/or the mixture needs to be maintained under stirring for at least 30 minutes.

If injectable formulations in the form of ready-to-use solutions are to be prepared in step a) of the aforesaid procedure, water for injectable preparations is used; after step d) an additional step, e), is undertaken in which the solution obtained in step d) is sterilized by filtering through a 0.22 μm filter.

If instead injectable formulations in the form of lyophilizates to be reconstituted in water on use are to be prepared, the process of the invention comprises a step, e), in which the solution obtained in step d) is sterilized by filtering through a 0.22 μm filter followed by a step, f), in which the solution obtained in step e) is subjected to lyophilization in a sterile environment.

Before undertaking the lyophilization, the solution is preferably diluted until a progesterone-hydroxypropyl-β-cyclodextrin complex is obtained whose concentration is less than 27% w/w. As will be shown in examples 4 and 5 to follow, the present inventors have in fact found that by lyophilizing solutions at higher concentrations, powders are obtained which dissolve in water over very long periods of time, in the order of 30-40 minutes, while from the solution diluted in accordance with the invention a product is obtained which can easily be reconstituted in water, resulting in a clear solution being obtained in 5-10 minutes.

The present inventors have surprisingly found that lyophilization treatment also enables stability in water of the progesterone-hydroxypropyl-β-cyclodextrin complex to be further increased.

In this respect the solution obtained by reconstituting the lyophilizate has an even greater stability than that of the solution obtained following the maturation process. The present invention also relates to unit dosage forms for intramuscular or subcutaneous administration of progesterone consisting of formulations in accordance with the present invention containing a quantity of progesterone between 25 and 100 mg.

A further advantage of the formulations of the invention is that the injectable solutions of the present invention enable concentrations of plasma progesterone to be achieved that are 3 times greater than those achieved with oil dispersions of the known art for the same dosage, thus enabling the dosages of progesterone required to achieve effective plasma concentrations to be considerably reduced. Moreover, as indicated in example 6, the present inventors have also found that lyophilization alone, when undertaken in the absence of the maturation process, also results in an increase in stability in water of the progesterone-hydroxypropyl-β-cyclodextrin complex. As already noted by the present inventors, in preparing lyophilizates of matured solutions, in order to obtain a lyophilizate which can be reconstituted in water within adequate time periods, lyophilization must be carried out on a solution in which the concentration of the progesterone-hydroxypropyl-β-cyclodextrin complex is less than 27% w/w.

Therefore, a further aspect of the present invention is a process for preparing an injectable progesterone formulation comprising the following steps:

a) preparing a solution of the progesterone-hydroxypropyl-β-cyclodextrin complex having a concentration less than 27% w/w;

b) lyophilizing the solution.

In accordance with a particularly preferred embodiment of the present invention the solution of step a) is prepared by a process that comprises the following steps:

a′) preparing an aqueous solution of hydroxypropyl-β-cyclodextrin;

a″) adding progesterone under stirring to the solution of step a′) preferably in a quantity equal to 10% by weight on the quantity of hydroxypropyl-β-cyclodextrin and optionally filtering to obtain a clear colourless solution;

a′″) if the solution obtained has a concentration greater than 27% w/w, diluting it with water for injectable preparations until the progesterone-hydroxypropyl-β-cyclodextrin complex concentration is less than this amount, then subjecting it to lyophilization.

The present invention will be better illustrated by the experimental examples which follow.

### EXAMPLE 1

48 g of hydroxypropyl-β-cyclodextrin (degree of substitution equal to 0.63; 0.5% unsubstituted β cyclodextrin content) are dissolved, under magnetic stirring, in about 47 g of deionised water. 4.8 g of progesterone are added to the transparent colourless solution under magnetic stirring, and the final weight is made up to 100 g with deionised water. It is left under stirring for about 40 minutes. The solution is filtered through a 0.45 micron filter to obtain a transparent colourless solution. The progesterone titre, effected by UV analysis, is found to be 55.52 mg/ml.

The solution thus obtained is maintained at 5° C. for 8 days. Under these conditions a white precipitate forms which is removed from the solution by filtering through a 0.45 micron filter.

A transparent colourless solution is obtained, found to be stable at 25° C. for at least 40 days.

The progesterone titre, effected by UV analysis, is found to be 52.54 mg/ml.

### EXAMPLE 2

48 g of hydroxypropyl-β-cyclodextrin (degree of substitution equal to 0.59; 0.6% unsubstituted β-cyclodextrin content) are dissolved, under magnetic stirring, in about 47 g of deionised water. 4.8 g of progesterone are added to the transparent colourless solution under magnetic stirring, and the final weight is made up to 100 g with deionised water. It is left under stirring for about 40 minutes. The solution is filtered through a 0.45 micron filter to obtain a transparent colourless solution. The progesterone titre, effected by UV analysis, is found to be 54.44 mg/ml.

The solution thus obtained is maintained at 5° C. for 3 days. Under these conditions a white precipitate forms which is removed from the solution by filtering through a 0.45 micron filter.

A transparent colourless solution is obtained, found to be stable at 25° C. for at least 40 days.

The progesterone titre, effected by UV analysis, is found to be 51.37 mg/ml.

### EXAMPLE 3

3 solutions are prepared in parallel by dissolving in 47.2 g of deionised water under magnetic stirring, for each of these solutions, 48 g of hydroxypropyl-β-cyclodextrin containing respectively 0.6% (sol. 1), 0.5% (sol. 2) and less than 0.1% (sol. 3) of unsubstituted β-cyclodextrin. When the solutions are transparent and colourless 4.8 g of progesterone are added and are then left under stirring for about 40 minutes.

The solutions obtained are clarified through a 0.45 micron filter to obtain a solution free of solid bodies. The clear solutions are maintained at a temperature of 5° C. for 15 days. Under these conditions a white precipitate forms after 1 day for solution 1 and after 4 days for solution 2 giving rise to solution cloudiness whereas, in the case of solution 3, no precipitate or cloudiness is found. Solutions 1 and 2 are filtered through a 0.45 micron filter after maturing for 15 days at 5° C. All three solutions obtained are transparent and colourless and are stable at 25° C. for at least 40 days.

### EXAMPLE 4

48 g of hydroxypropyl-β-cyclodextrin (degree of substitution 0.63, 0.5% unsubstituted β-cyclodextrin content) are dissolved, under magnetic stirring, in 47.2 g of deionised water. When the solution is transparent and colourless, 4.8 g of progesterone are added and left under stirring for about 40 minutes.

The solution obtained is clarified through a 0.45 micron filter to obtain a solution free of solid bodies. The clear solution is maintained at a temperature of 4-6° C. for 8 days. Under these conditions a white precipitate forms which is removed from the solution by further filtration with a 0.45 micron filter.

The solution thus obtained is dispensed in vials to the extent of 1.1 g/vial and then, after stoppering, placed in the lyophilizer chamber and subjected to the lyophilization process. The lyophilizate obtained is compact and ivory-white in colour.

The sample thus obtained is treated with 1 ml of water for injection and forms a clear solution after 30 minutes without applying any stirring.

The progesterone titre, effected by UV analysis on the reconstituted product, is found to be 32.75 mg/g, equal to 49 mg/vial.

### EXAMPLE 5

Two 40 g samples of a matured solution, prepared as described in example 4, are diluted respectively with 60 g (Sol. A, dilution 1→2.5) and 40 g (Sol B, dilution 1→2) of deionised water maintaining the solution under magnetic stirring for 10 minutes.

The two solutions thus prepared are split in parallel into vials to the extent of 2.5 g/vial and of 2 g/vial. After stoppering, the vials are inserted into the lyophilizer chamber and subjected to the lyophilization process, to obtain ivory-white coloured compact lyophilizates.

The lyophilizates obtained are reconstituted utilizing 1 ml of water for injection per vial. The time needed for complete dissolution, with the lyophilizates obtained from both diluted solutions, was found to be under 10 minutes.

The progesterone titre, effected by UV analysis on the solutions prior to lyophilization, was found to be 46 mg/vial in the case of solution A and 46.5 mg/vial in the case of solution B. The UV analyses on the reconstituted product confirm the concentrations of both samples.

The lyophilizate, following reconstitution with 1 ml of water for injection, is found to be clear and colourless without precipitate formation for at least 50 days at 25° C. and at least 5 days at 5° C.

The results obtained demonstrate that the dilution of the solution prior to lyophilization provides a considerable reduction in the time needed for lyophilizate reconstitution.

### EXAMPLE 6

6a) A solution of progesterone and hydroxypropyl-β-cyclodextrin is prepared as described in example 4. However, instead of maintaining the solution at low temperature for 8 days, vial filling and lyophilization are carried out directly as described in example 4.

The lyophilizate obtained is compact and ivory-white in colour.

The sample thus obtained, treated with 1 ml of water for injection, forms a clear solution after 30 minutes without applying any stirring.

The progesterone titre, effected by UV analysis on the reconstituted product, is found to be 34.7 mg/g, equal to 52 mg/vial.

6b) A solution of progesterone and hydroxypropyl-β-cyclodextrin is prepared as described in example 4, but omitting the maturation stage. 40 g of the non matured solution is diluted with 60 g of water and vial filling and lyophilization are carried out directly, as described in example 4.

The lyophilizate obtained is compact and ivory-white in colour.

It is reconstituted using 1 ml of water for injection.

The time needed for complete dissolution of the buffer was found to be under 10 minutes.

The progesterone titre, achieved by UV on the solution prior to lyophilization, is found to be 47.3 mg/2.5 g of solution and the lyophilizate concentration, reconstituted with 1 ml of water for injection, is found to be 30.9 mg/g equal to 46.3 mg/vial.

After reconstituting with 1 ml of water the lyophilizate is found to be clear and colourless without a precipitate forming for at least 20 days at 25° C. and at least 5 days at 5° C. After this period the formation of a precipitate is observed which in the space of 24 hours adheres to the base of the vial.

The result obtained in example 6b shows that direct lyophilization of hydroxypropyl-β-cyclodextrin solutions, even in the absence of the solution maturation process, leads to formulations being obtained which, once reconstituted in water, give rise to solutions with improved stability compared with the initial solutions.

1. Injectable progesterone formulation comprising a complex between progesterone and hydroxypropyl-β-cyclodextrin, characterised by containing a quantity of unsubstituted β-cyclodextrin less than 0.3% w/w on the quantity of hydroxypropyl-β-cyclodextrin.

2. Formulation as claimed in claim 1 wherein the quantity of unsubstituted β-cyclodextrin is less than 0.1% w/w on the quantity of hydroxypropyl-β-cyclodextrin.

3. Formulation as claimed in claim 1 suitable for intramuscular or subcutaneous use.

4. Formulation as claimed in claim 1 in the form of a ready-to-use aqueous solution.

5. Formulation as claimed in claim 1 in the form of a lyophilizate for the extemporaneous preparation of an injectable aqueous solution.

6. Process for preparing a formulation as claimed in claim 1 comprising the following steps:
a) preparing an aqueous solution of hydroxypropyl-β-cyclodextrin having a concentration between 12% and 55% w/w;
b) adding progesterone under stirring to the solution of step a) and optionally filtering to obtain a transparent colourless solution;
c) maintaining the solution at a temperature between 2 and 8° C. for a period of at least 2 days;
d) carrying out a clarification filtration using a filter with a porosity between 0.45 and 0.8 micron.

7. Process as claimed in claim 6 wherein in step a) the hydroxypropyl-β-cyclodextrin concentration is 48% w/w.

8. Process as claimed in claim 6 wherein in step b) the progesterone is added in a quantity equal to 10% by weight on the quantity of hydroxypropyl-β-cyclodextrin.

9. Process as claimed in claim 6 wherein in step b) the progesterone is added in micronized form.

10. Process as claimed in claim 6 wherein in step c) the solution is maintained at a temperature of 2-8° C. for a period between 2 and 10 days.

11. Process as claimed in claim 10 wherein in step c) the solution is maintained at a temperature of 2-8° C. for a period between 3 and 8 days.

12. Process as claimed in claim 11 wherein in step c) the solution is temperature controlled at 5° C.

13. Process as claimed in claim 6 wherein said formulation is in the form of a ready-to-use aqueous solution, water for injectable preparations is used in step a) and, after step d), a further step e) is undertaken in which the solution of step d) is sterilized by filtering through a 0.22 μm filter.

14. Process as claimed in claim 6 wherein said formulation is in the form of a lyophilizate for the extemporaneous preparation of an injectable solution and after step d) a step e) is undertaken in which the solution of step d) is subjected to sterilization by filtering through a 0.22 μm filter followed by a step f) in which the solution is lyophilized in a sterile environment.

15. Process as claimed in claim 14 wherein in said step f), before carrying out the lyophilization, the solution obtained in step e) is diluted with water until a concentration less than 27% w/w of the progesterone-hydroxypropyl-β-cyclodextrin complex is obtained.

16. A unit dosage form for intramuscular or subcutaneous progesterone administration consisting of a formulation as claimed in claim 1 containing progesterone in a quantity between 25 and 100 mg.

17. Process for preparing an injectable progesterone formulation comprising the following steps:
a) preparing a solution of the progesterone-hydroxypropyl-β-cyclodextrin complex having a concentration less than 27% w/w;
b) lyophilizing the solution.

18. Process as claimed in claim 17 wherein step a) comprises the following steps:
a′) preparing an aqueous solution of hydroxypropyl-β-cyclodextrin;
a″) adding progesterone under stirring to the solution of step a′) and optionally filtering to obtain a clear colourless solution;
a′″) diluting the solution if necessary with water for injectable preparations.

19. Process as claimed in claim 18 wherein in step a″) the progesterone is added in a quantity equal to 10% by weight on the quantity of hydroxypropyl-β-cyclodextrin.

20. Injectable formulation obtained from the process claimed in claim 17.
